(19) United States
(12) Patent Application Publication
(10) Pub. No.: US 2012/0214121 A1
(43) Pub. Date: Aug. 23, 2012

(54) Orthodontic Treatment Integrating Optical Scanning and CT Scan Data

(75) Inventor: Alex M. Greenberg, New York, NY (US)

(73) Assignee: Greenberg Surgical Technologies, LLC, New York, NY (US)

(21) Appl. No.: 13/359,246

(22) Filed: Jan. 26, 2012

Related U.S. Application Data
(60) Provisional application No. 61/436,514, filed on Jan. 26, 2011.

(51) Int. Cl. A61C 13/34; A61C 7/00; A61B 6/03

(52) U.S. Cl. 433/24; 378/4; 433/213

(57) ABSTRACT

A process for creating a dental model to avoid periodontal defects during planned dental work includes obtaining CT scan data and optical scan data of a patient's dentition and integrating the CT scan data and the optical scan data by at least one of surface to surface registration, registration of radiographic markers, and registration of optical markers of known dimensions, to produce a dental model that includes the dentition and underlying bone and root structures. The process then segments anatomic sites of the tooth roots and underlying bone. A plan for the dental work is then generated based on the segmented anatomic sites, whereby the plan avoids periodontal defects based on the knowledge of the anatomic sites of the roots and underlying cortical bones in the dental model.

### CROSS-REFERENCE TO RELATED APPLICATIONS

This application claims priority from U.S. Provisional Application Ser. No. 61/436,514, filed Jan. 26, 2011, the contents of which are hereby incorporated by reference in its entirety.

### BACKGROUND OF THE INVENTION

1. Field of the Invention

The present invention relates to the creation of integrated Computed Tomography (CT) and optical scan data for customized orthodontic treatment planning and the production of custom dental models, custom indirect orthodontic brackets, and custom orthodontic aligners that prevent periodontal and mucogingival defects. More specifically, the present invention relates to an integration of a CT scan and an optical scan of dentition derived from an oral cavity or a dental model to prevent the penetration of cortical bone of a jaw in the alveolar region by the tooth root as a consequence of orthodontic movement.

2. Description of the Related Art

In the process of orthodontically applied biomechanical forces on teeth, an orthodontist utilizes different appliances which can be fixed to the teeth or removable, or a combination of fixed and removable appliances. Depending on the appliance used, the teeth are moved in certain directions and at a certain rate. Teeth are able to move through the alveolar bone as result of the biodynamic process of the periodontal ligaments that attach the tooth root to the bone. When force is placed to move the root forward this creates pressure on the forward moving aspect of the root with resultant osteoclastic activity that resorbs the bone allowing the movement and tension on the back of the root with resultant osteoblastic activity and bone deposition. Depending on the type of movement there can be variations of tension and pressure along the root surface causing bone resorption and deposition as the tooth is moved to its desired position. If this movement is excessive or does not properly take into account the alveolar bone morphology, penetration of the bony cortices can occur with resultant gingival recession and mucogingival defects. Gingival recession occurs at the superior aspect of the root in the attached gingiva where there is a decrease in the attached gingiva and exposure of the root surface. A mucogingival defect is a more advanced lesion where the attached gingiva is completely receded and only the areolar mucosa remains which creates a more advanced loss of periodontal attachment and root exposure. A mucogingival defect with its lack of keratinized gingival is less cleansable and less resistant to the effects of bacteria in plaque which causes an inflammatory response that creates a worsening of the periodontal defect and allows the gingival recession and bone dehiscence to deteriorate which jeopardizes the health of the tooth and can cause its premature loss. This is a greater problem in adult orthodontics than orthodontics in children and adolecesents. However, if the orthodontic treatment in children and adolescents is not performed correctly, areas of root perforation or penetration with dehiscence can lead to periodontal disease in these patients in their adult years. Endodontic lesions can also develop as a result of orthodontic tooth movement which can relate to tooth collisions, overcompression of the periodontal ligament, penetration or perforation of the bony cortices that lead to devitalization of the tooth with loss of apical vascular supply to the pulp, or development of root resorption.

As a result of orthodontic movement of teeth, the tooth roots can penetrate a supporting alveolar cortical bone and result in the dehiscence of the bone overlying the root. FIG. 1A shows a cross section of a tooth 10 situated in its supporting alveolar cortical bone 20. The tooth 10 has a crown 11 and a root 12. A superior end 12a of the root 12 faces the crown 11 and an apex 12b of the root 12 is distal from the crown. In FIG. 1A, the right side 24 of the alveolar cortical bone 20 faces externally toward a subject's cheek or lips and is referred to as the buccal cortex or labial cortex, respectfully. The left side 22 of the alveolar cortical bone 20 faces internally toward the tongue and is referred to as the lingual cortex. FIGS. 1B, 1C, 1D are simplified schematic diagrams of the cross section of FIG. 1A and illustrate different movements of the tooth that can cause dehiscence. FIG. 1B shows tipping about an apex of the root, FIG. 1C shows a lateral movement without tipping, and FIG. 1D shows a tipping about a central portion of the tooth. FIGS. 2-5 show different general forms of the alveolar bone anatomy.

There are numerous scientific articles that document the problem in orthodontics that when teeth are not moved correctly that the roots can perforate or penetrate the cortical bone whether on the buccal/labial or lingual/palatal aspects. More specific examples of tipping, rotation, bodily movement, intrusion and extrusion tooth movements are described in the following paragraphs.

Tipping occurs a root pivots around an axis of rotation created by the orthodontic appliance. Depending on the tipping motion the axis of rotation may be in the inferior (FIG. 6A), middle (FIG. 6B), or superior (FIG. 6C) ⅓ of the root. If, for example, a tooth is tipped lingually about an axis in the middle or superior third of the root, then there is the possibility for the root apex to perforate the buccal/labial cortex of bone as shown in FIG. 7. In orthodontics there is a tension and pressure side to the tooth root depending on the biomechanical force exerted upon it. On the pressure side osteoclastic activity is stimulated within the periodontal ligament, while on the tension side osteoblastic activity is stimulated. FIG. 8 shows that if a tooth is tipped where the apex is moved labially at the inferior aspect, then ostetoclastic activity will occur in the inferior labial aspect, and osteoblastic activity in the superior labial ⅓. On the lingual aspect there is tension on the superior ⅓ with osteoclastic activity and pressure on the inferior lingual side with osteoblastic activity. In the areas where tipping causes osteoclastic activity there will be bone resorption. Depending on the thickness of the alveolar cortical bone, which is usually thinner on the buccal/labial aspect that the lingual/palatal aspects, it is possible as a result of this resorptive process that the root can perforate the bone so as to create a simple perforation at the apex as shown in FIG. 7, which will not create a periodontal defect, but can weaken the tooth as result of less bone support and cause devitalization if the nutrient vessel at the apex of the tooth is disrupted and cause the need for endodontic treatment.

FIG. 9 shows that if perforation of the cortical bone occurs on the superior aspect of the root and bone interface, then a root dehiscence is created whereby the loss of periodontal attachment causes gingival recession and if there is complete loss of keratinized gingival which ranges in height from 2-6 or more mm, then a mucogingival defect will occur that also compromises the integrity of the tooth cause it to be weakened. Adequate periodontal attachment is necessary to withstand the functional occlusal forces that are placed on teeth during the masticatory process. Mucogingival defects usually require free gingival grafting, pedicled grafting or other allografts in order to replace the keratinized gingival. Bony perforations can be repaired with bone grafting with or without membrane placement for guided tissue regeneration.

Referring now to FIG. 10, rotational orthodontic movement occurs in the axial plane. Rotational movements can cause cortical perforation or dehiscence because of the root anatomy. As teeth rotate it is possible that a line angle of the root can perforate the bone on the pressure side of the movement opposite to the tension side.

Bodily movement occurs in the sagittal plane when the tooth moves without any tipping as shown in FIG. 11. If bodily movement of a tooth is too much then it can perforate or penetrate the cortical bone depending on the method of treatment. Bodily movement will have the potential for a greater degree of root dehiscence and an associated mucogingival defect due to the greater amount of root exposure through the cortical bone.

Intrusion occurs when the root is forced apically along its longitudinal axis as shown in FIG. 12. This type of movement can cause a root to perforate through the bone depending on the alveolar bone shape, for example in obtuse mandibular plane to incisal angulations, such as skeletal bimaxillary protusion. In cases where there is a significant undercut in the bony alveolus, the intrusion of the root can penetrate the labial cortical bone in such a movement.

Extrusion occurs when a tooth is forced coronally along its longitudinal axis as shown in FIG. 13. Depending on the alveolar bone form and the root anatomy, i.e., bulbous root, flared root, trifurcated premolars, accessory rooted molars, it is possible in a extrusive movement that a tooth could perforate or penetrate the superior cortical plate on either the labial or lingual side.

Any combination of the above movements could result in the perforation or penetration of a root apex through the cortical bone and cause a resultant periodontal defect.

Because of the above, it is desirable in orthodontics to avoid root perforation of the bone cortex to prevent loss of periodontal attachment, a complication of orthodontics. Presently, the fabrication of orthodontic aligners through a series of virtual created stereolithographic models such as Align Technology's method on U.S. Pat. Nos. 5,975,893; 6,699,037; 6,722,880; and 201000167243, do not account for the root anatomy. These aligners are created solely based on the anatomy of the crowns from dental molds and impressions that are CT scanned and optically scanned for software planning. A further prior art U.S. Pat. No. 7,241,142 does account for root anatomy and considers a method for incorporating root anatomy. However this patent does not teach orthodontic tooth movements based on the full tooth anatomy (crown to root) that takes into account the relationship of the tooth root to the cortical alveolar bone and the potential for root penetration and perforation of the cortical bone and the prevention of gingival recession, mucogingival defects, and endodontic lesions.

U.S. Pat. No. 6,319,006 teaches bonding a Shape of Known Dimensions” on the dentition that allows acquisition in the CT and optical scan of the dentition which allows registration. The limitation of this method relates to the need to apply physical markers of standardized known dimensions and shape to the teeth. EP1486900 (A1) teaches the surface to surface registration of surfaces which has limitations based on the inaccuracies of plaster dental casts and presence of scatter artifact in the CT scan image from the presence of dental restorations.

The integration of Cone Beam CT and optical scan data by the superimposition of 3D images based on surface geometry is taught by US patent application publications 20090316966 and 20100151405, in which matching of surfaces may or may not be distorted by scatter artifact. These patent publications do not teach the prevention of periodontal defects and resultant gingival recession, mucogingival defects, and endodontic lesions.

### SUMMARY OF THE INVENTION

An object of the present invention is to avoid periodontal defects during dental work.

According to an embodiment of the present invention, a process for creating a dental model to avoid periodontal defects during planned dental work includes obtaining CT scan data and optical scan data of a patient's dentition and integrating the CT scan data and the optical scan data by at least one of surface to surface registration, registration of radiographic markers, and registration of optical markers of known dimensions, to produce a dental model that includes the dentition and underlying bone and root structures. The process then segments anatomic sites of the tooth roots and underlying bone. A plan for the dental work is then generated based on the segmented anatomic sites, whereby the plan avoids periodontal defects based on the knowledge of the anatomic sites of the roots and underlying cortical bones in the dental model.

The plan for the dental work may be a plan for orthodontic tooth movements. Thicknesses of the underlying cortical bones are taken into account, and the plan allows no more than a 50% penetration through the thicknesses of the cortical bones by the tooth roots during the orthodontic tooth movements. Relative positions of the roots and the cortical bones may be displayed during the step of determining a plan.

The process includes generating equipment to implement at least a portion of the dental work. The equipment may comprise orthodontic equipment to implement planned tooth movements such as, for example, orthodontic aligners. Alternatively, the equipment generated may be a surgical guide. In this case, the step of obtaining CT scan data includes CT scanning a radiographic template including a module for the surgical guide, such that the dental model includes the position of the module relative to the tooth roots and the cortical bone. A drill guide sleeve is created in the module based on the position of the module relative to the tooth roots and the cortical bone after the step of generating a plan.

The dental work to be planned may, for example, also include orthognathic surgery, in which case the equipment generated is surgical splints. The equipment generated may alternatively be a dental implant, a dental prosthesis, or a Temporary Anchorage Device (TAD).

According to one embodiment of the invention, the step of obtaining comprises obtaining a CT scan of a radiographic template and a patient when the radiographic template is in a patient's mouth, the radiographic template having a negative impression of the patient's dentition and a surface of known dimensions, obtaining a CT scan of the radiographic template alone, and obtaining an optical scan of the radiographic template with the surface of known dimensions.

According to this embodiment, the step of integrating comprises integrating the CT scan of the radiographic template and the patient with the CT scan of the radiographic template alone by registering radiographic markers, and integrating the optical scan and the CT scans by registering the surface of known dimensions. The radiographic template may further include a module to be used during dental surgery. The module is formed into a surgical template based on the anatomic sites of the roots and underlying cortical bones after the step of generating a plan. Furthermore, the module and template may be modular components in that the module is connectable to and releasable from the template by interlocks and the surface of known dimensions is connectable to and releasable from one of the template and the module by further interlocks.

According to other embodiments of the present invention, the dental work to be performed may be one of oral and maxillo facial surgery.

An embodiment of the process is to allow the creation of dental models by stereolithography, rapid printing, rapid prototyping methods. These dental models will have accurate representations of the teeth including undercuts as well as the dental anatomy of tooth roots. These representations of the tooth roots can be colored in a different color than the rest of the dental model. A series of these dental models can be produced by rapid prototyping so as to create a series of orthodontic aligners for a series of planned tooth movements for the correction of various orthodontic malocclusions. This is an improvement over the method utilized by Align Technologies based on U.S. Pat. Nos. 5,975,893, 6,699,037, 6,722,880, 201000167243 which details the process which creates a CT scan of a dental cast using a CT industrial scanner. In such a process that manipulates the CT image of the teeth and the undercuts to create stereolithographic models of each stage of the planned orthodontic tooth movement. These individual stereolithographic models are then utilized to create dental aligners on an industrial scale production line.

Alternatively, the dental model may be a virtual model.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings, wherein like reference characters denote similar elements throughout the several views:

FIG. 1A is a tooth in a cortical bone;

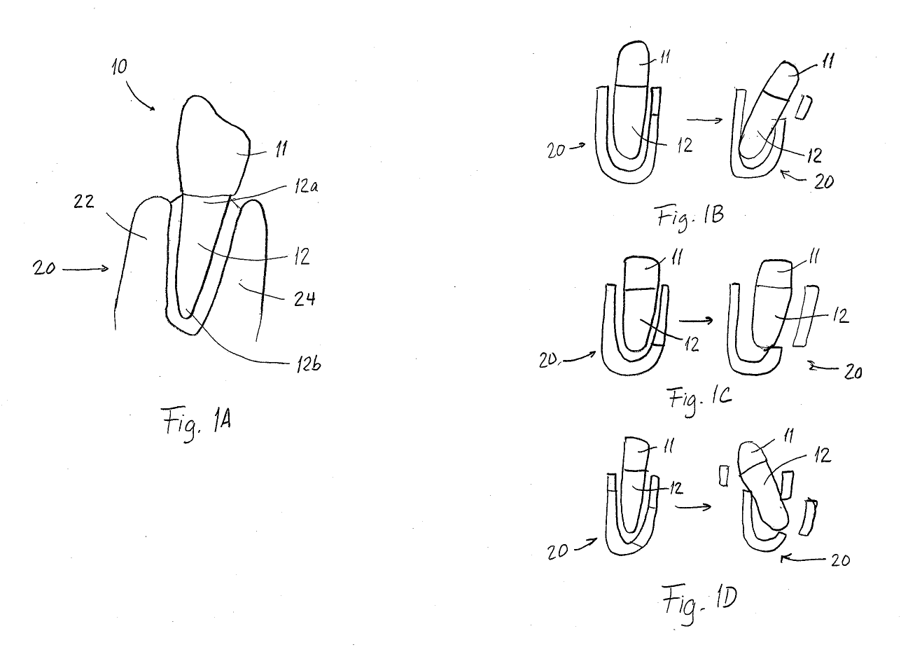

FIGS. 1B-1D are schematic diagrams showing tooth movements;

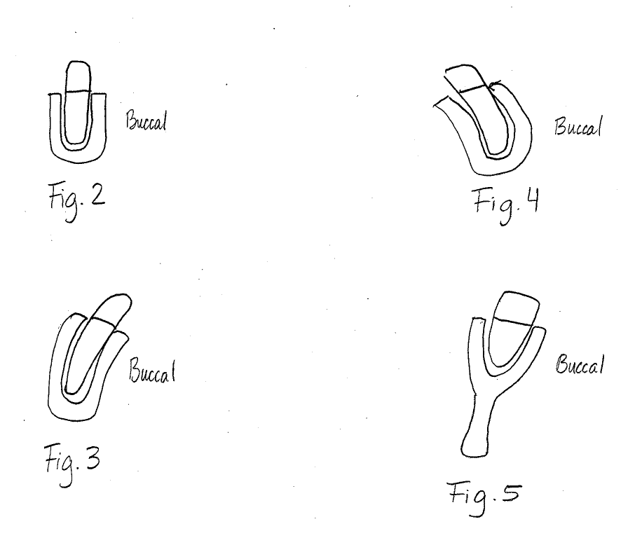

FIGS. 2-5 are schematic diagrams showing various configurations of corical bones;

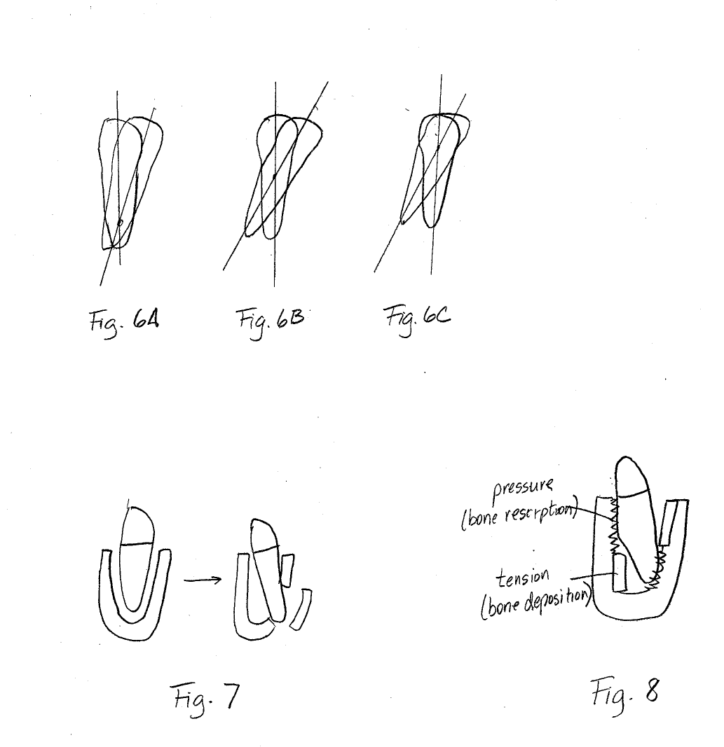

FIGS. 6A-6C are schematic diagrams showing tooth rotation about different axes;

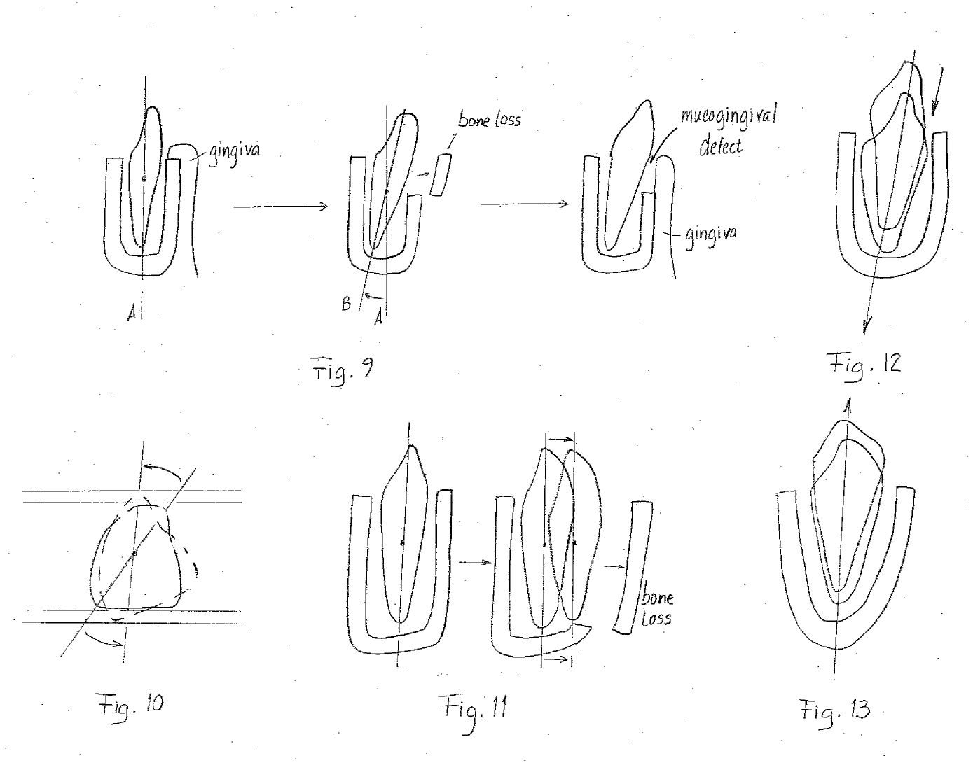

FIG. 7 is a schematic diagrams showing a tipping movement of a tooth;

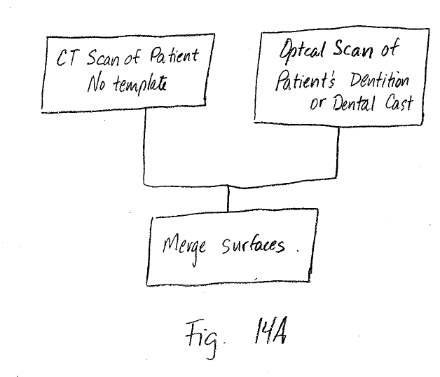

FIG. 8 is a schematic diagram showing bone resorption and bone deposition during movement of a tooth;

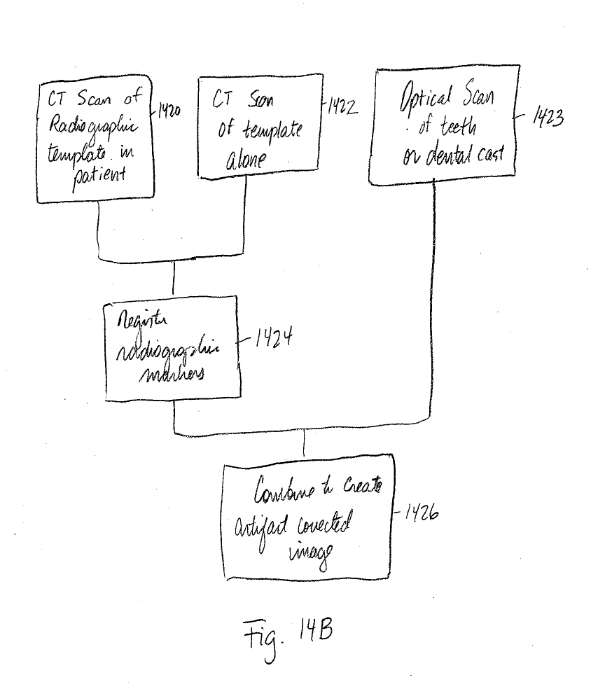

FIGS. 9-13 are schematic diagrams showing various tooth movements;

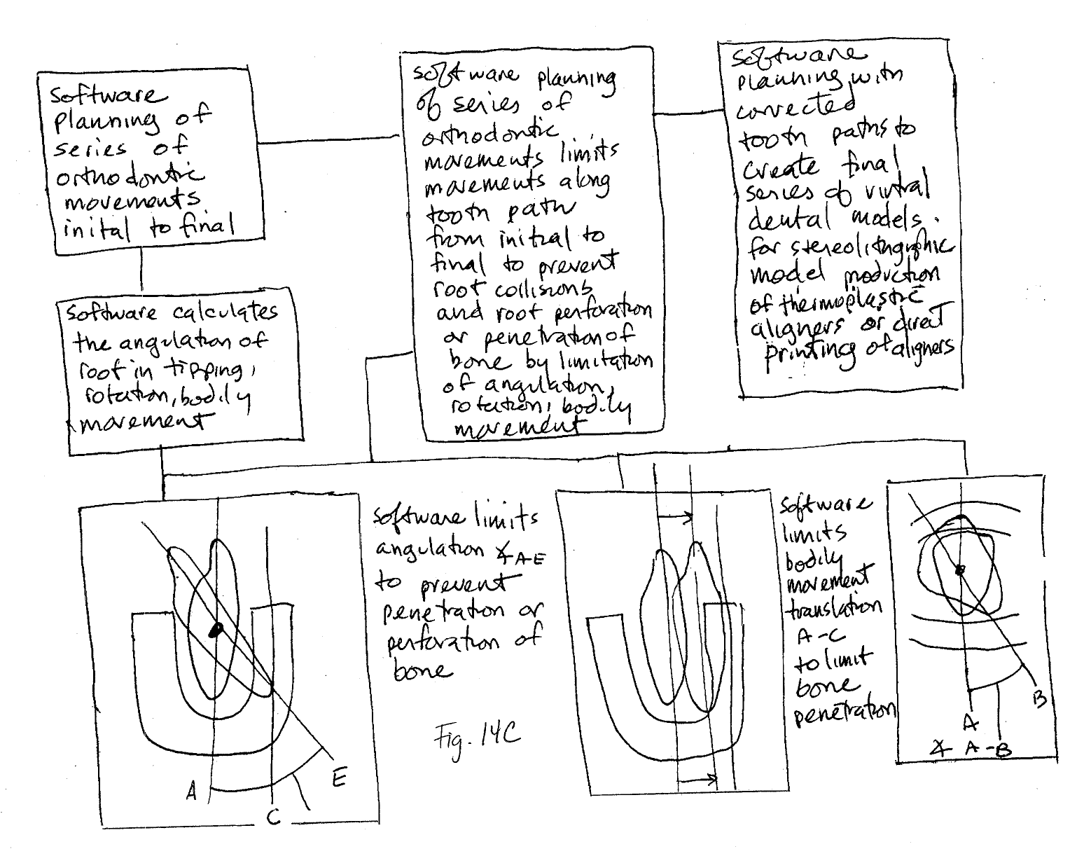

FIGS. 14A-14C are flow diagrams showing method of integrating CT scans and optical scans;

FIGS. 15A-15C are schematic diagrams showing steps in creating a radiographic marker on a tooth with a orthodontic bracket;

FIG. 16 is a schematic diagram showing an allowed perforation of the cortical bone;

FIGS. 17A-17C are schematic diagrams of a tooth tilt and subsequent rotation;

FIG. 18 is a schematic diagram showing a bone penetration and a bone graft;

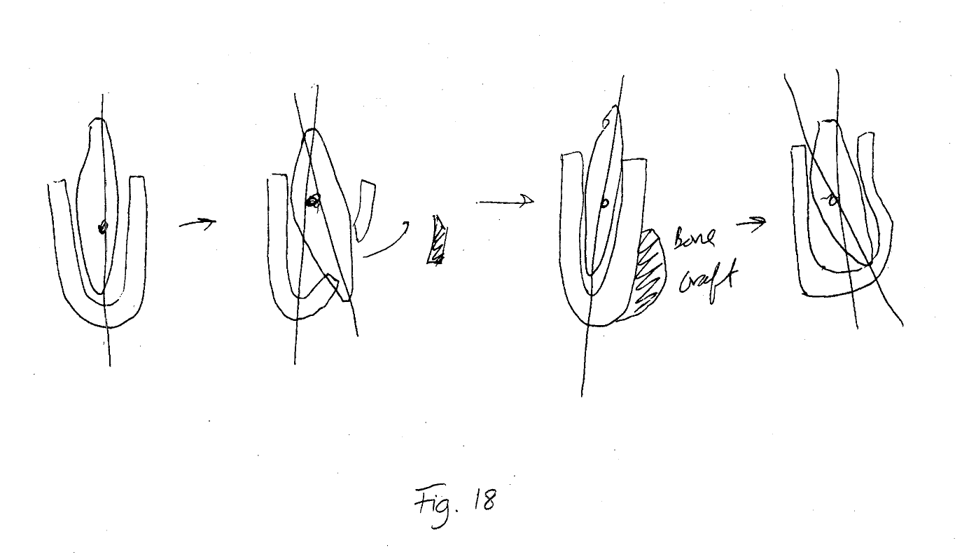

FIG. 19 is a schematic diagram of a template that can be used during a CT scan of a patient;

FIG. 20 is another embodiment of a template that can be used during a CT scan of a patient;

FIG. 21 is a further embodiment of a template that can be used during a CT scan of a patient;

FIGS. 22A-22F are schematic diagrams showing steps for forming a dental cast with a surface of known dimensions;

FIGS. 23A-23C are schematic diagrams of further templates that are used for two-sided impressions of a patient's dentition; and

FIG. 24 is a schematic diagram illustrating a crown to be inserted onto a dental implant using the claimed invention.

### DETAILED DESCRIPTION OF THE PRESENTLY EMBODIMENTS

According to an embodiment of the present invention, a combined scan data showing both a dentition and underlying bone and root structures is created by integrating CT scan data and optical scan data. The integration may be accomplished by one of the following three methods.

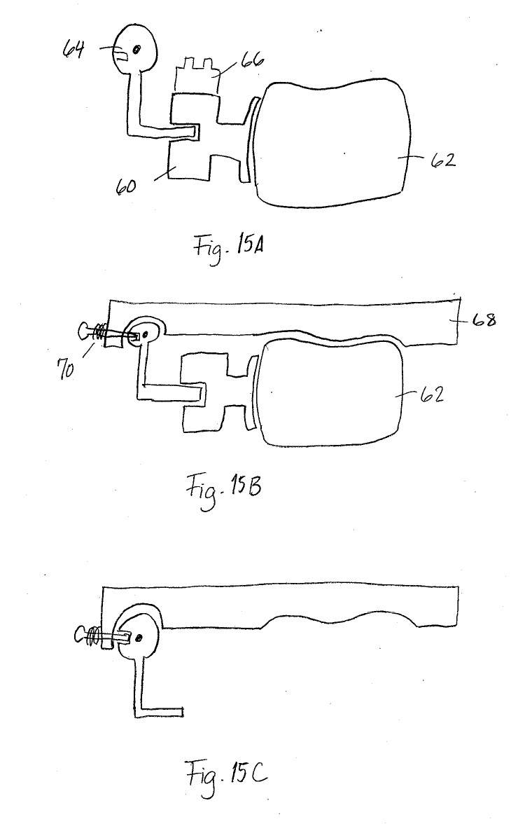

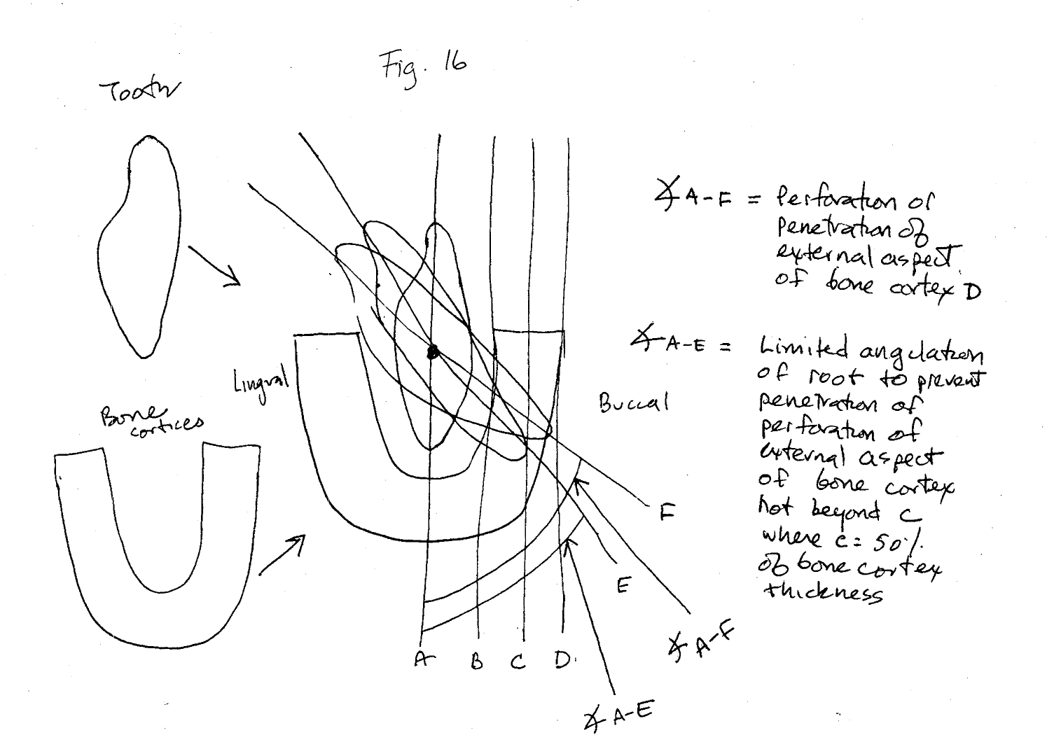

According to a first method of the present invention shown in FIG. 14A, an optical scan of the teeth 1400 and a CT scan of the patient is made 1402 and a superimposition and registration with a 3-dimensional (3D) volume rendering of the dentition based on matching of selected surfaces of the teeth 1404 to produce an artifact corrected image. This is less difficult when there are no dental restorations in the teeth that could create scatter artifact. It may be necessary to use a dental appliance such as an orthodontic bracket as disclosed in U.S. patent application Ser. No. 13/299,269, which can be scanned by both CT and optical scan to allow registration of the two datasets, as it contains a radiographic marker and a shape of known dimensions (SKD). FIG. 15A shows an orthodontic bracket 60 mounted on a tooth 62, i.e., a molar. The orthodontic bracket 60 of FIG. 15A has a radiographic marker 64 and an SKD 66. Here the radiographic marker 64 has a definite positional relationship with the SKD 66 and both have a definite relationship with the tooth 62. A radiographic template 68 is arranged at a specific relationship relative to the tooth 62 in FIG. 15B. The radiographic marker 64 is connected to the radiographic template by a spring-loaded retentive member 70. There are many variations of orthodontic brackets made of ceramic, plastic, and metal. The metal brackets cause scatter artifacts and are not suitable for a combined optical and radiographic marker. However, ceramic or plastic brackets can be used for this purpose because they do not cause scatter artifacts. Orthodontic brackets such as Edgewise or Roth prescription, which have extended hooks from the orthodontic wire holding portion may also have an SKD that contains a radio dense marker. These shapes also have known dimensions that can be contained within the software for recognition by surface or contour by a suitable algorithm.

According to a second method of creating the combined scan data shown in FIG. 14B, a CT scan is taken of a patient having a radiographic template in a patient's mouth 1420 that has an SKD and 6 radiographic fiducial markers. The radiographic template has a negative impression of the dentition and is CT scanned separately 1422 and optically scanned 1423. The two CT scans are combined by matching radiographic markers 1424. The three data sets, i.e., the CT scan of the patient, the CT scan of the appliance, and the optical scan of the impression or dental cast are combined to form an artifact corrected image 1426. The template may be an impression tray having a single handle with radiographic markers incorporated therein and an SKD as an optical marker oriented toward the impression side. As an alternative, the template may be a double handled in which each handle has an SKD oriented 180 degrees from the other, i.e., one facing the impression tray side and one facing away from the impression tray. The SKD facing away from the impression tray may be detachable and could be attachable to the dental cast by a stem so that when the model is poured, the dental cast will contain the SKD in the correct relation to the dentition as it was in the CT scan. A reliable representation of the dentition and the SKD as it was situated relative to the patient, which allows a cast of the dentition and the SKD to be scanned optically to create a data set for merger with CT data of the radiograph template containing the SKD. The radiographic template may also be formed as a double tray to obtain a simultaneous impression of the upper and lower jaws.

According to the third embodiment, a CT scan of a patient with a radiographic guide containing 6 radiographic markers and a separate scan of the radiographic guide and the registration of the two data sets using the radiographic markers creates a virtual dental model consisting of the bone and dentition. The clean data set of the radiographic guide provides a negative impression that can be reversed by software to provide an artifact corrected image of the dentition.

The above three embodiments allow merger of CT data and optical scan data of the dentition to create a virtual dental model that incorporates and uses CT data of tooth roots, which in turn allows prevention of the perforation buccal or linguistic surfaces of the cortical alveolar bone and thereby prevents periodontal and mucogingival defects and endodontic lesions/root resorption.

By using software analysis of the cortical bone and its relationship to the root during planning of orthodontic movements as shown in FIG. 14C, it is possible to avoid the excessive resorption of bone by osteoclasts through the tension and pressure of orthodontic biomechanics. In many cases the labial and lingual cortices of the bone can be very thin, especially in the anterior mandible and the maxilla. Roots of teeth such as molars have furcations and premolars have root concavities and furcations that can also be problematic when there is bone resorption and loss of periodontal attachment. The software analysis according to the present invention performs this function after the integration by registration of the CT and optical scan data to create an improved virtual dental model. The software proceeds to segment the root shape from the bony cortex. These segmented anatomic sites will be subject to analysis during the simulated planned tooth movements to achieve orthodontic treatment through aligners or fixed orthodontics.

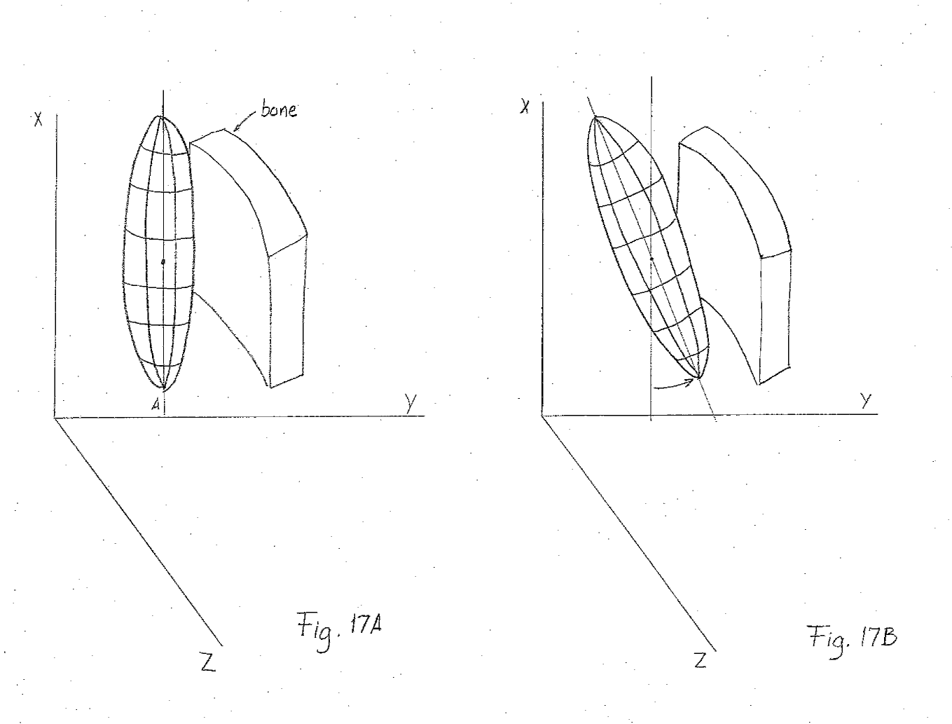

FIG. 16 depicts the segmented tooth and bone cortices. Line A extends longitudinally through the tooth. Lines B, C, D are parallel lines that represent the inner side, the center, and the outer side of the buccal bone cortex. The software analysis prevents the root from perforating the bony cortex based on an analysis of the thickness and shape of the buccal and lingual/facial and palatal alveolar bone morphology based on an algorithm that calculates the angulation of the movement of the tooth by tipping, rotation, bodily, intrusion, and extrusion movements and compares it to an angulation of the root to the cortical bone that creates a limitation of the movement in that stage of a planned series of orthodontic tooth movements. A thickness of the cortical bone is also taken into account whereby for example the root cannot penetrate more than 50% of the cortical bone, i.e., cannot penetrate past line C in FIG. 16. The dentist will be notified by the software that an undesirable movement and possible bony perforation can occur and the movement will be negated as a “no go zone”. The software and the root outline will be different colors in a 2 dimensional sagittal view which can also be viewed in a 3D mode simultaneously in real time in the x, y, and z axes.

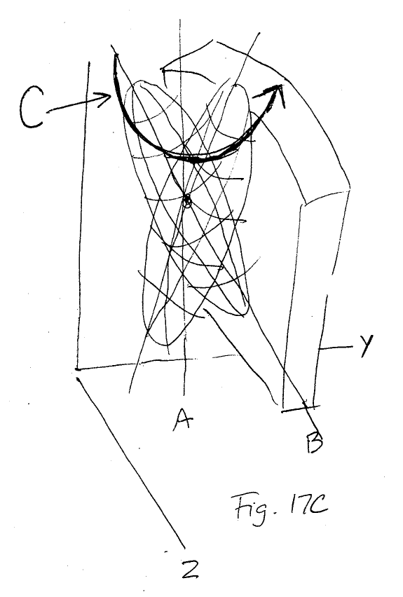

Three dimensional images also allow better viewing of “round tipping”. Round tipping is a 3 dimensional rotational tipping movement that is useful for moving teeth in a such a way that collisions with other teeth can be avoided for example. FIGS. 17A-17C show an example of round tipping. The tooth in FIG. 17A is tipped from longitudinal axis A to longitudinal axis B shown in FIG. 17B. Finally, the tipped tooth of FIG. 17C is rotated about axis A as indicated by the arrow C in FIG. 17C. Such a movement will be prevented by the software which will have to determine a different series of movements to achieve the desired tooth movement. The clinician will be able to see an animation displaying the series of desired orthodontic tooth movements in all three orthogonal directions with the appropriate amounts of biomechanical forces applied as the series of planned tooth movements occur according to the various steps of the treatment and to ensure that unwanted or undesirable effects on the tooth root and local bony anatomy will not occur as per the clinician's original prescription for treatment. This animation and series of tooth path movements can be interactive and iterative with the clinician so that modifications of the treatment plan can be implemented. A certain amount of bone cortex resorption can be allowed also based on the Hounsfield units which can be utilized to determine the “hardness of the cortical bone” and its suitability for the resorptive process of orthodontics as it relates to the thickness of the cortex and desired movement. The software will then cause a modification in the planned series of tooth movements in order to achieve the desired result. Such information could also allow the software to make a clinical recommendation to the orthodontist to have bone grafting performed to thicken the bone as in the method of Wilkodontics: Wilcko W M, Wilcko T, Bouquot J E, Ferguson D J. Int J Periodontics Restorative Dent. 2001 February; 21(1):9-19. Rapid orthodontics with alveolar reshaping: two case reports of decrowding.

As a result of the present invention, each model of an aligner that will be fabricated by mass industrial stereolithography will be created containing the root anatomy information that has accounted for this special consideration of tooth movement limitation based on the CT scan information that has been integrated into the planning process so that cortical bone penetration or perforation, or endodontic damage that could create periodontal recession, mucogingival defects or endodontic lesions. This is also of particular importance in cases where there are missing teeth where tooth movement is being performed for positioning teeth more ideally for fixed bridge or dental implant restorations. It may also reveal a limitation of pure orthodontic aligner treatment whereby orthodontic brackets may need to be placed on teeth for conventional orthodontic treatment or placement of orthodontic brackets for elastic traction combined with the aligner design such as hooks incorporated into the aligner or other special attachments that promote certain rotations, intrusions, tipping, rotation or bodily movements or other biomechanical force. If such orthodontic appliances are metallic they can create scatter artifact and difficulty in merging the CT and optical scan data and in such cases an alternative method is described below.

This method can also be of particular importance in patients with dentofacial deformities and skeletal malocclusions which comprise 3% of the population (Bell W, Proffit W, White R: Surgical Correction of Dentofacial Deformities, W.B. Saunders 1980). These individuals have dental compensations as result of their maxillomandibular dysplasia and skeletal jaw deformities that may well result in extreme malpositions of teeth with especially thin cortices or highly deformed alveolar bone anatomy that creates issues related to typical orthodontic movements that could result in thinning of bony cortices, potential for root perforations, root resorption, possible need for bone grafting, other corticotomies for accelerated or more favorable toot movement based on the anatomy. Such patients could possibly have already developed root dehiscences or cortical bone perforations as a result of their more unusual anatomy. In the series of virtual models that may be created in presurgical orthodontics where it is desirable to segment the jaws for surgery, this method also becomes critical to create the correct alignment of roots for segmentation and to prevent cortical perforation, spacing between roots, the avoidance of buccal cortex perforation from molar torquing that occurs in reestablishing the root positions within alveolar bone so that surgical movements may be performed.

Hounsfield units play a role in this process by helping to determine the bony cortex density and the risk for thinning and root perforation and the degree of biomechanical force that will be needed to move the root through the cortical plate.

Align Technology fabricates a series of the clear plastic retainers, or “aligners”, that sequentially move the teeth at a rate of 0.25-0.33 mm every 14 days. The aligners should be worn at least 20 hours per day, but are taken out for meals and for brushing and flossing. The number of aligners needed for a particular case depends on the extent of tooth movement required.

Various further uses of these improved virtual and rapid manufactured/rapid printed dental models can be derived such as the creation of orthodontic aligners by a direct rapid printing method that includes the undercuts, creation of orthodontic aligners based on a vacuum formation of a web of thermoplastic material on a series of stereolithographic models that represent the planned stages of orthodontic treatment, indirect methods for placement of orthodontic brackets for fixed orthodontic appliance treatment (as taught by U.S. Pat. Nos. 6,099,314; 6,976,840; 7,147,464; and 7,738,989), which can also include surgical drill guides for the placement of dental implants and TADs (Temporary Anchorage Devices) for orthodontics. This method further improves upon the Align Technologies methods disclosed in U.S. Pat. Nos. 5,975,893, 6,699,037, 6,722,880, 201000167243 and Cadent methods disclosed in U.S. Pat. Nos. 6,099,314, 6,976,840, 7,147,464, and 7,738,989, including the dental root anatomy into the virtual plan of the patient which allows the planned series of orthodontic tooth movements to prevent root collisions, alveolar cortical bone penetration and perforations, endodontic lesions, gingival recession and mucogingival defects. In this way when the teeth are moved by software manipulation of the virtual image of the patient, the tooth movements whether they are rotational, tipping, intrusion, extrusion, bodily movements in the correction of Class I, II, III tooth crowding, Class I, II, III overjet and overbite discrepancies, combinations of using cut outs in the aligners for Class II and III elastics, or orthodontic brackets for elastic traction, tooth attachments with particular shapes to promote rotation, extrusion, tipping, or bodily movements. Knowledge of root anatomy can also affect the desired velocity of movement and pattern movement in order to avoid collision between roots and the creation of periodontal defects and endodontic lesions in the process of tooth movement by the planned biomechanical movement of the aligners. There are considerable variation in the pattern of tooth roots that cannot be estimated only by extrapolation from the longitudinal axis of teeth as taught by US Patent Application Publication 20100167243. The incorporation of CT data allows more precise knowledge of tooth root anatomy into this type of removable aligner treatment for orthodontic malocclusion as taught by U.S. Pat. No. 7,241,142, which does not teach the avoidance of alveolar bony cortex penetration and perforation so as to prevent the development of gingival recession, mucogingival defects, or endodontic lesions. The integration of the CT data and optical scan data of the tooth crown anatomy including the undercuts allows a more precise dental model to be created where the teeth are represented by accuracy to within 100 microns or less. This allows a superior aligner to be fabricated that incorporates the CT data of the root anatomy into the biomechanical model of planned orthodontic tooth movements. With the accumulation of a data base of cases treated that it would be possible to create a database of common root anatomy patterns that coincide with different anatomic tooth forms and classifications of dental malocclusion such as Class I, II and III. This would also facilitate the ability to treat more surgical cases of skeletal malocclusion with aligners as there is a greater understanding of the complete dental anatomy and bony anatomy of maxillomandibular dysplasia.

It may also be possible that direct printing of aligners based on the virtual model may be possible using rapid printing, rapid prototyping technologies by the additive and subtractive processes of the software manipulation of the scanned radiographic appliance into the correct form of an aligner that would be made of a malleable material with adequate flexibility to fit over the undercuts of teeth.

The orthodontic aligners could also incorporate planning for dental implants and the creation of combined orthodontic aligners with a surgical drill guide template for the placement of dental implants based on the planned final orthodontic position of teeth and the planned location and trajectory of a dental implant. In the series of orthodontic aligner treatments it would also be possible to use the orthodontic aligner as a drill guide for the planned placement of Temporary Orthodontic Anchorage devices (TADS) that may be part of elastic traction, hybrid aligner and fixed banded orthodontic treatment, and surgical cases where TADS of varying sizes could be used for surgical correction of dentofacial deformities/maxillomandibular dysplasia. The integration of CT data and optical scan data for treatment planning and the creation of a surgical guide would allow the prevention of root damage from TAD placement.

Once CT scan data or cone beam CT (CBCT) scan data and optical scan data have been registered by replication of surface geometries a complete virtual model is created. In this virtual model the following dental relationships based on the local coordinates is achieved. A treatment planning environment for orthodontics can be achieved. As result of such planning, a 3D model is created in fact a series of 3D models with complete crown and root data is created and can be printed by stereolithography or rapid prototyping (RP) to have a series of physical models on which either orthodontic aligners can be fabricated, or orthodontic brackets can be placed for indirect bonding of brackets.

There are also dental prosthetic considerations that often have to be considered in coordination with orthodontic treatment. Such dental prosthetic considerations include conventional fixed bridges or implant retained prostheses whether fixed or removable overdentures. Orthodontic patients who are children or adolescents may be missing teeth due to congenital absence or loss due to caries and endodontic infections, and rarely from juvenile periodontitis. In such cases the orthodontic treatment may need to take into consideration the movement of teeth to position teeth more ideally for tooth preparation for conventional prosthetics. Virtual tooth preparation can be incorporated in the planning and when the teeth are ideally located a new optical scan of the tooth preparation whether by direct or indirect impression means can be obtained and registered to the data set to create an updated data set. In cases where dental implants are planned whether in adolescents or adults, coordinated dental implant placement with a temporary dental prosthesis can be planned. A CAD CAM prosthesis can be created to fit within the dental model as certain orthodontic goals are achieved. This dental model can be pin indexed or segmented and such a model can allow hybrid treatment delivery such as partial treatment with orthodontic aligners, Temporary Anchorage Devices, or dental implants to be incorporated in the treatment planning environment. In orthognathic surgical intervention a series of presurgical, surgical and post surgical interventions can be planned and simulated virtual surgical splint can be created that is then fabricated by CAD CAM processes. In late adolescent and adult patients undergoing orthognathic surgery the dental prosthetic plan who are partially edentulous the dental prosthetic plan whether for conventional or implant retained prosthetics can have such treatment planning incorporated into the surgical planning. Surgical splints for maxillomandibular fixation of the osteotomy segments during surgery can be created based on articulated virtual models, and segmented movements as it relates to any fixed orthodontic appliances. In minimally invasive orthognathic surgery such surgical guides could be fabricated in whole or in modular fashion so as to allow the supramucosal application of rigid fixation hardware for fixation of the bone segments in the desired planned position. These surgical splints can also incorporate drill guides for the placement of TADS or dental implants in such partially edentulous patients undergoing orthognathic surgery.

The integration of CBCT and optical scan data to create an articulated set of models for planning all aspects of orthodontics, oral and maxillofacial surgery, and creation of surgical splints for orthognathic surgery is known such as, for example, the Medicim Maxilim software. However this software does not include planning for removable orthodontic aligners or the creation of models for the indirect placement of orthodontic brackets, or the prevention of alveolar bone cortex penetration/perforation by tooth roots during orthodontic treatment to prevent the development of gingival recession, mucogingival defects, or endodontic lesions. Furthermore such software does not involve dental prosthetic planning or the incorporation of a CAD CAM milled dental prosthesis or implant or TAD surgical guide fabrication.

Orthodontic movement for periodontal prosthesis and CAD CAM virtual design of temporary prosthesis demonstrates the importance of root anatomy as due to the periodontal bone loss in that in these patients with periodontal disease there can be loss of crestal bone and exposure of the furcations of root surfaces of premolars and interradicular furcas of molar teeth. These teeth can also have a greater degree of mobility due to the loss of periodontal attachment. Orthodontic treatment may be necessary in these patients to reposition teeth back into the dental arch, uprighting of molars when adjacent teeth have been lost, positioning of teeth for fabrication of a periodontal prosthesis, incorporation of dental implants into a treatment planning. In such cases the planned tooth movement may need to be integrated with periodontal treatment such as bone grafting and guided tissue regeneration, TADS, dental implants, fabrication of CAD CAM temporary dental prosthesis for conventional fixed or dental implant retained or a combination of these dental prosthetics. There can be an integration of dental implant prosthetic parts such as posts as well so that after implant placement a prefabricated post can be placed, or a direct optical or conventional impression of the final placed implant with a scan body abutment is performed from which a final post can be fabricated.

It may also be possible that rather than actually producing the stereolithography models that a printing process can print each custom series of aligners based on the virtual 3D models. This would save considerable cost in making the stereolithography models and avoid waste of vacuum forming material currently used on a web with excess material unused. The addition of material onto the virtual dental model occlusal surfaces of teeth including the undercuts would allow the printing of these aligners by rapid printing processes. The aligners would be created after the subtraction of the segmented virtual model.

According to an embodiment of the present invention, the software will evaluate a thickness of the segmented image of the cortical bone in the sagittal, axial, and coronal planes as it relates to the root outline. There will be a limitation of the amount of cortical bone that can be resorbed by the planned movement so that there will be the avoidance of perforation at either the inferior labial or superior lingual or superior labial and inferior lingual depending on the degree of final movement or interim movement to be created by the orthodontic appliance forces or the thickness of the bone or its density. For example, during a tipping movement a mathematical algorithm will evaluate and compare the angulation of the root in the longitudinal axis A and compare it to the angulation as it tips lingually to lines E and F in FIG. 16, where F is where the root fully penetrates the cortex to the exterior side D and E is where the root penetrates 50% of the cortex to the center C, and the angulations will be calculated. The software will limit the angulation to which the root can be tipped so that E will not exceed 50% penetration of the cortical bone as a limiting step of the planned orthodontic tooth movement from the initial to the final tooth path. Angle E will be less than Angle F based on a mathematical calculation. In cases where Angle F is necessary the software can advise pretreatment bone grafting to increase the amount of cortical bone. Hounsfield units can be utilized as well to determine the density of the cortical bone and its susceptibility to orthodontic forces. These different factors can be evaluated so that the software can determine the limitation of the orthodontic movement that can be performed based on the crown to root anatomy. Pixilated images in the sagittal 2D view will have limitations placed on the number of pixels that may be subtracted, and the same in the 3D volume rendering as to the number of voxels that can be removed to allow the root's movement. In the process of planning the desired orthodontic tooth movements the root anatomy in its 2D sagittal and 3D images, there would be a recognition by the software of the loss of bone continuity of pixels and voxels that could be aided by the Hounsfield units depending on the density of the bony cortex sagittal section. This could also be determined in the axial plane which also allows the definition of the root to bone thickness. A discontinuity of the voxels of the bony cortex would create a “no go” zone for the tooth root which would prevent the movement of the root through the bony cortex or to collide with another tooth root as described above and in the virtual planning and creation of a series of dental models incorporating the full tooth anatomy from crown to root apex and the adjacent segmented bony cortices. This would be calculated on a per tooth basis as the alignment algorithm formula that would cause the incisal and occlusal cusps of the teeth to be correctly aligned within the arch and as it related to the opposing arch by articulation of the opposing dental arches. The clinician will be able to see an animation displaying the series of desired orthodontic tooth movements in all three orthogonal directions with the appropriate amounts of biomechanical forces applied as the series of planned tooth movements occur according to the various steps of the treatment and to ensure that unwanted or undesirable effects on the tooth root and local bony anatomy will not occur as per the clinician's original prescription for treatment. This animation and series of tooth path movements can be interactive and iterative with the clinician so that modifications of the treatment plan can be implemented. A 3D aligner can be virtually created on top of the virtual dental model which includes the undercuts and a series of custom orthodontic aligners can be created that will be printed from a malleable material that is flexible and allows placement over the teeth in a series of aligners that cause the planned orthodontic movement to occur. A similar process can be performed when there has been a radiographic template used in the scan that allows an artifact corrected image to be created from which through additive and subtractive processes a series of orthodontic aligners can be created as whole templates or in a modular manner.

According to an embodiment of the present invention, segmentation of the bone cortices and contours and root shapes is utilized to limit the allowed orthodontic movements in the fabrication of orthodontic aligners for the prevention of root/bone perforations in the creation of orthodontic aligners. It can also allow in adults with periodontal disease Class I, II with mild to moderate periodontal bone loss to reposition the teeth back into the confines of the cortical bone to then permit healing of the periodontal defect by the periosteum or permit improved bone and grafting and guided tissue regeneration.

This application discloses an improved method that incorporates the disclosure of U.S. patent application Ser. No. 13/299,269 by reference. The present invention permits the combined registration of radiographic markers and a shape of known dimensions (i.e. Lego such as in Med3D, Heidelberg, Germany) to allow the registration of the optical data of a CT scan and optical scan data as an improvement over U.S. Pat. No. 6,621,491. Furthermore another limitation of U.S. Pat. No. 6,621,491 is the dependence of determination of the dental implant trajectory to the surface of adjacent teeth. In contrast, the method according to the present invention utilizes a tooth form or dental bridge (fixed partial denture) form in coordination with the underlying bony anatomy to determine the desired dental implant trajectory. This application improves further upon U.S. application Ser. No. 13/299,269 and U.S. Pat. No. 6,621,491 by the segmentation of the root and alveolar bone cortices as described above to prevent the penetration and perforation of alveolar bone cortices as a result of orthodontic treatment and thereby the prevention of gingival recession, mucogingival defects, and endodontic lesions in cases where orthodontics is coordinated with dental implant treatment and the creation of surgical guides for the placement of dental implants and can include the placement of temporary CAD CAM milled temporary dental prosthetics. There are cases where depending on the materials used, these temporary dental prosthetics may be considered as permanent.

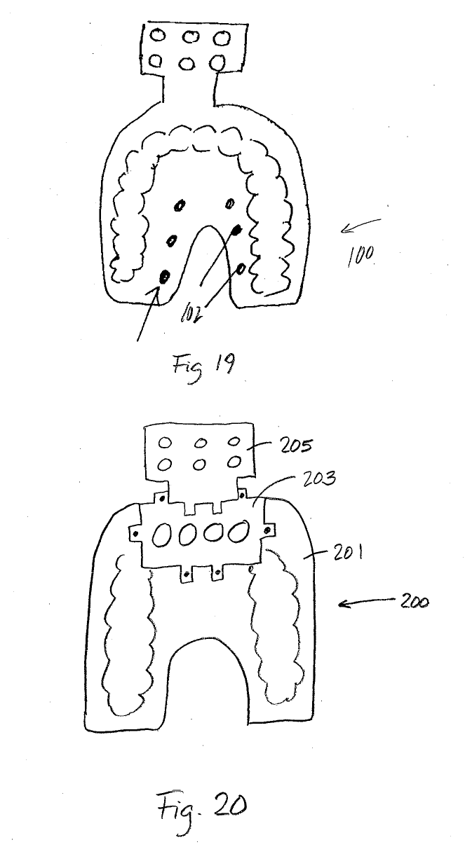

R Jacobs et al “Predictability of a three dimensional planning system for oral implant surgery Dentomaxillofac Rad 1999 28: pp 105-111, and Van Steenberghe “A custom template and definitive prosthesis” Int J Maxillofac Implants 2002:17: pp 663-70 and U.S. Pat. No. 7,574,025 use a dual scan process of a radiographic template with fiducial markers. U.S. Pat. No. 5,967,777 allows the registration of the radiographic appliance scan and in a patient's mouth and then a separate scan of the appliance in a Styrofoam box, thus creating two data sets that allow the creation of an artifact corrected image. FIG. 19 shows an example of a radiographic appliance 100 that may be used. According to this embodiment, the appliance includes a template with fiducial markers 102. An SKD 104 such as a Lego extends external to a patient's mouth when the template is in the mouth. Using this device, a CT scan can be taken of the appliance in the patient and outside of the patient as mentioned above to create and artifact corrected image. The two data sets digitized images are merged in the planning software with registration and superimposition of the radiographic template to the bone images. The fiducial markers can be made of a radiodense material such as, for example, metal filings or gutter percha. This scan appliance can be a standardized template manufactured from a injection molded material that contains in its handle the SKD. This appliance can also be made in a modular form in accordance with US Patent application 20060291968, the content of which is incorporated herein by reference.

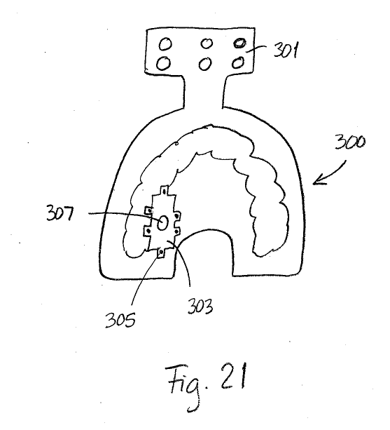

FIG. 20 is another example of appliance 200 which may be used. According to this embodiment, the appliance includes a template 201, a drill template module 203, and an SKD as separate parts. The three parts are connected together via interlocks 205, 207. In this case, the drill template module includes fiducial markers. A CT scan is performed of the interconnected parts in the patient. An optical scan is taken of the appliance and the two datasets are registered. The drill template module can be worked on to create a drill guide using known methods, while taking into consideration the location and configuration of the tooth roots. The drill template module is reconnected with the template using the interlocks and the template 201 is placed in the user's mouth. The drill template module is now usable to drill the teeth and place implants or a temporary bridge.

For all of the above embodiments, the shape of known dimensions can be either a positive or a negative depending upon the needs of the software manipulating the data for the processing of the digitized image in order to create the registration between the data sets of the CT of the patient+radiographic appliance and the radiographic appliance, and then a separate additional registration of an optical scan of the negative impression of the radiographic appliance and shape of known dimensions. The negative impression contains the occlusal surfaces of the teeth in a custom malleable material such as dental acrylic or can be a negative impression of the teeth with a material such as a polyether or poly vinylsiloxane applied to the radiographic appliance which has been used as a dental impression tray. The radiographic appliance can be a standardized form in different sizes to accommodate different sized mouths such as small, medium, and large.

U.S. Pat. No. 7,574,025 discloses a method for creating an oral implant drilling template and is incorporated herein by reference. The merger of the two data sets described above allows an artifact corrected image to be superimposed over the bone image so that in the presence of dental restorations or fixed metal orthodontic appliances the radiographic appliance can be segmented in a correct relationship to the anatomic bony structures. This allows the insertion of a post segmentation functional element such as a dental implant trajectory that can be transformed into a shape within the radiographic template clean artifact corrected image as drill trajectory channel that will be formed as a subtraction of material in the rapid prototyping/rapid printing of the digitally created surgical drill guide template.

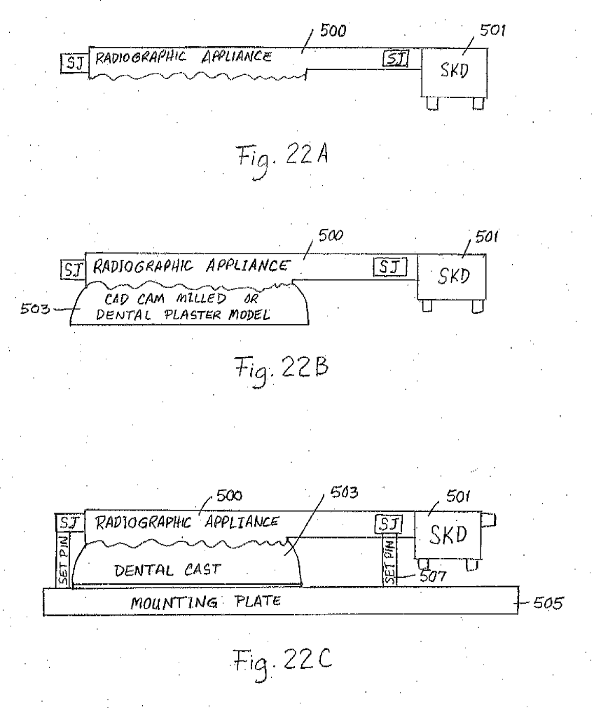

The radiographic scan appliance can then be optically scanned by either a hand held scanner (Sirona CEREC, 3M Lava, D4D E4D, Densys, Cadent iTero) or desk top scanner (3MLava, Straumann Etkon, D4D E4D) to create a virtual negative impression of the negative impression. The STL file of the optically scanned virtual model of the dentition can then be registered with the CT scan data of the registered patient data+radiographic guide and the artifact corrected radiographic guide through the registration of the shape of known dimensions. In this way an artifact corrected image of the dentition can be represented in the combined CT scan of the patient+radiographic guide data. This integrated CT scan and optically scanned data images can then allow a multitude of treatment planning options for the practitioner within a virtual environment that can allow a variety of outputs through rapid manufacturing/rapid printing/rapid prototyping and CAD CAM manufacturing methods. Such outputs can be for the fabrication of dental implant surgical guides, jaw fracture bone plating drill guides, medical applications such as electrode insertion for modulation of the Sphenopalatine/nasoplatine ganglion for vascular effects as per U.S. Pat. Nos. 7,120,489 and 7,729,759, and orthodontic appliances. FIG. 21 illustrates creation of a Computer Aided Design/Computer Aided Manufacturing (CAD/CAM) created part for Sphenopalatine Ganglion (SPG) guidance of an electrode. According to the embodiment, a template 300 with an impression tray includes an SKD 301 and a module 303 for SPG guidance for an electrode placement. The module 303 has fiducial markers 305. A CT scan is made of the device in the patient and an optical scan is made of the device 300. The guidance trajectory 307 is then made based on the combined scan data. Such fabrication processes can also be performed in modular forms in which the SKD 301 connects to the module 303 via interlocks and the module and/or the SKD connect to the template via interlocks. This virtual planning environment can also allow the virtual insertion of crowns, bridges, dental implant fixed and removable prostheses and their parts for the planning, fabrication, and insertion of dental prosthetics, dental implants, orthodontic aligners and any combination of these for dental treatment.

An embodiment of this process is to allow the creation of dental models by sterolithography, rapid printing, rapid prototyping methods. These dental models will have accurate representations of the teeth including undercuts as well as the dental anatomy of tooth roots. These representations of the tooth roots can be colored in a different color than the rest of the dental model. A series of these dental models will then be produced by rapid prototyping so as to create a series of orthodontic aligners for a series of planned tooth movements for the correction of various orthodontic malocclusions. This is an improvement over the method utilized by Align Technologies based on U.S. Pat. Nos. 5,975,893, 6,699,037, 6,722,880, and US Patent Pub. 201000167243, for example, which create a CT scan of a dental cast using a CT industrial scanner. In such a process that manipulates the CT image of the teeth and the undercuts to create stereolithographic models of each stage of the planned orthodontic tooth movement. These individual stereolithographic models are then utilized to create dental aligners on an industrial scale production line. The method of the present invention is an improvement because it obviates the need for a creating a dental cast that has to be separately CT scanned and instead uses the optical scan data of the digital impression to be merged with the CT scan of the patient+radiographic template and separate scan of the radiographic template to create the virtual model of the patient. The incorporation of CT data allows more precise knowledge of tooth root anatomy into this type of removable aligner treatment for orthodontic malocclusion as taught by U.S. Pat. No. 7,241,142, which does not teach the avoidance of alveolar bony cortex penetration and perforation so as to prevent the development of gingival recession, mucogingival defects, or endodontic lesions. This method according to the present invention further improves upon the Align Technologies method in the virtual plan of the patient which allows the planned series of orthodontic tooth movements to include the segmented root and alveolar bone cortices anatomy. In this way when the teeth are moved by software manipulation of the virtual image of the patient, the tooth movements whether they are rotational, tipping, bodily movements in the correction of Class I, II, III tooth crowding, Class I, II, III overjet and overbite discrepancies, combinations of using cut outs in the aligners for Class II and III elastics, or orthodontic brackets for elastic traction, tooth attachments with particular shapes to promote rotation, extrusion, tipping, or bodily movements. Knowledge of root anatomy can also affect the desired velocity of movement and pattern movement in order to avoid collision between roots and to avoid the penetration and perforation of the alveolar bone cortices to avoid the creation of periodontal defects such as gingival recession, mucogingival defects, and endodontic lesions in the process of tooth movement by the planned biomechanical movement of the aligners. It is well understood from dental anatomy studies and CT data concerning tooth roots that there are considerable variation in the pattern of tooth roots that cannot be estimated only by extrapolation from the longitudinal axis of teeth as taught by US Patent App. Pub. 20100167243. The incorporation of CT data allows more precise knowledge of tooth root anatomy into this type of removable aligner treatment for orthodontic malocclusion. The integration of the CT data and optical scan data of the tooth crown anatomy including the undercuts allows a more precise dental model to be created where the teeth are represented by accuracy to within 100 microns or less. This allows a superior aligner to be fabricated that incorporates the CT data of the root anatomy into the biomechanical model of planned orthodontic tooth movements. It would also be possible that with the accumulation of a data base of cases treated that it would be possible to create a database of common root anatomy patterns that coincide with different anatomic tooth forms and classifications of dental malocclusion such as Class I, II and III. This would also facilitate the ability to treat more surgical cases with aligners as there is a greater understanding of the complete dental anatomy and bony anatomy of the maxillomandibular dysplasia.

It may also be possible that direct printing of aligners based on the virtual model may be possible using rapid printing, rapid prototyping technologies as a digital subtraction of the scanned radiographic appliance into the correct form of an aligner or the application of virtual material onto the dental model so as to create the aligner that would be made of a malleable material with adequate flexibility to fit over the undercuts of teeth.

These orthodontic aligners could also incorporate planning for dental implants and the creation of combined orthodontic aligners with a surgical drill guide template for the placement of dental implants based on the planned final orthodontic position of teeth and the planned location and trajectory of a dental implant in coordination with a virtually planned CAD/CAM model of a temporary dental prosthesis. In the series of orthodontic aligner treatments it would also be possible to use the orthodontic aligner as a drill guide for the planned placement of Temporary Orthodontic Anchorage devices (TADS) that may be part of elastic traction, hybrid aligner and fixed banded orthodontic treatment, and surgical cases where TADS of varying sizes could be used for surgical correction of dentofacial deformities/maxillomandibular dysplasia.

This integrated CT and optical data set would also be useful for the creation of dental implant drilling templates that would use the planned dental prosthesis as a guide for the planned trajectory of the dental implant as opposed to simply relying on the anatomy of the surfaces of adjacent teeth as in the U.S. Pat. No. 6,319,006. In this way a virtual model of the patient can be created in which the radiographic template will be converted into a surgical drilling template that can then be created by either rapid manufacturing, rapid printing or CAD/CAM milling. Such treatment in specific cases may require coordination with orthodontic treatment whether by orthodontic removable aligners, fixed orthodontic appliances or a combination of them. As such the new method described in this patent application whereby there is segmentation of the root and alveolar bone cortical anatomy allows the creation of orthodontic aligners or indirect orthodontic bracket placement in coordination with planned CAD/CAM temporary dental prosthesis and dental implant placement with the ability to prevent the penetration or perforation of the alveolar cortical bone to prevent gingival recession, mucogingival defects or endodontic lesions.

Further embodiments of this methodology include various modular forms of manufacturing for combining optical scan data with CT scan data for the coordination of orthodontic treatment and dental implant placement based on the modular method of incorporating a CAD CAM virtual dental prosthesis into the virtual integrated treatment planning environment. This coordination of orthodontic aligner or indirect bracket placement treatment and dental implant placement based on the modular crown as opposed to relying on the adjacent tooth surfaces as in U.S. Pat. No. 6,319,006. An example would be the creation of a modular radiographic template as described above and shown in FIG. 20 in which the handle containing the SKD 205 interlocks by semiprecision or other types of attachments to a modular part of the radiographic modular template 203 which is also interlocked via semiprecision attachments into radiographic template framework part 201 to create a total radiographic template 200. Modular template 203 could, for example, be a temporary bridge prosthesis that has been CAD/CAM milled and is attached to the radiographic template framework part 201 so as to incorporate the final temporary prosthesis and by extension the shape of the final prosthesis into the CT scan data so as to provide the correct prosthetic information that will be used in the treatment planning of the dental implant trajectory or trajectories for the creation of a surgical drill template. A CT scan is performed of the patient with the custom radiographic template in the mouth. The radiographic template can be made of a malleable material such as dental acrylic or a polyvinylsiloxane or polyether impression made with the radiographic template which can be of a standardized form for different sized mouths small, medium, and large, for example, can be utilized. An optical scan of the total radiographic template 200 can be created using a desk top scanner or hand held scanner in a dental office or alternatively at a dental laboratory. The interlocked parts 203, 205 are removed from the total radiographic template 200 and the modular part 203 is scanned separately. The CT scan data sets of the patient+total radiographic template 200 and the modular part 203 are merged and registered via the radiographic/fiducial markers and a separate registration and merger of the optical scan data is created so as to create a integrated CT scan and optical scan virtual model of the patient. Planning for the dental implant trajectory is performed and a drill guide template modular part 203 is fabricated by rapid printing, rapid prototyping, and/or CAD/CAM milling with insertion of drilling sleeves and inserted back into radiographic template 201. If a polyvinylsiloxane or polyether impression material has been utilized then that material can be removed for the insertion of modular part 203 drill guide into the part 201 for clinical use. An alternative is to use the optical scanned model merged with the virtual model of the planned temporary bridge as would be obtained via virtual crown planning optical scan software systems such as hand held Lava, E4D, iTero, or desktop scanners such as Lava, Etkon, Everest, dental wings to create a surgical drill template via rapid printing, rapid prototyping or CAD/CAM milling that would have the accuracy of the fit of the occlusal surfaces from the optical scan of the impression and the CT scan data of the bony anatomy. In this way a modular method of fabricating a surgical drill template could be achieved in strictly virtual space with the fabrication of the surgical drill template. It is possible that utilizing a optical scan of the patient's dentition by a hand held scanner that a radiographic template in whole or modular form could be created for the CT scan and then that same data set could be utilized through the integrated merge of the CT scan and optical scan data of the radiographic template that has a SKD that a dental implant surgical drill template could be created. The same data sets could also be integrated with planned orthodontic aligner so that in cases where there would be combined orthodontic treatment and dental implant treatment that coordination between these different treatment aspects of the patient can be planned by a single practitioner or communicated between different dental practitioners who may be generalists or specialists. Such planning could be further incorporated and integrated into dental practice management systems for the total management of these cases within a dental office, other dental offices, in coordination with dental laboratories.

A further embodiment of this method would be for the insertion of an electrode through the greater palatine foramen into the vicinity of the sphenopalatine gangion (SPG) also known as the nasopalatine ganglion (NPG) for the modulation of electrofrequency to cause vasodilation of the cerebral vasculature in patients suffering from stroke or dementia as per U.S. Pat. Nos. 7,120,489, 7,729,759, 7,561,919, 7,640,062. Such an integration of the CT scan of the patient+radiographic template containing the SKD and a separate scan of the total template or the modular part of the template merged via registration of an optical scan of the radiographic template would allow the creation of a surgical template to allow the insertion of the electrode via the greater palatine foramen.

A further embodiment of this method is to include use of a modular surgical drill template for the insertion for the insertion of a temporary CAD/CAM milled crown with a post on a dental implant.

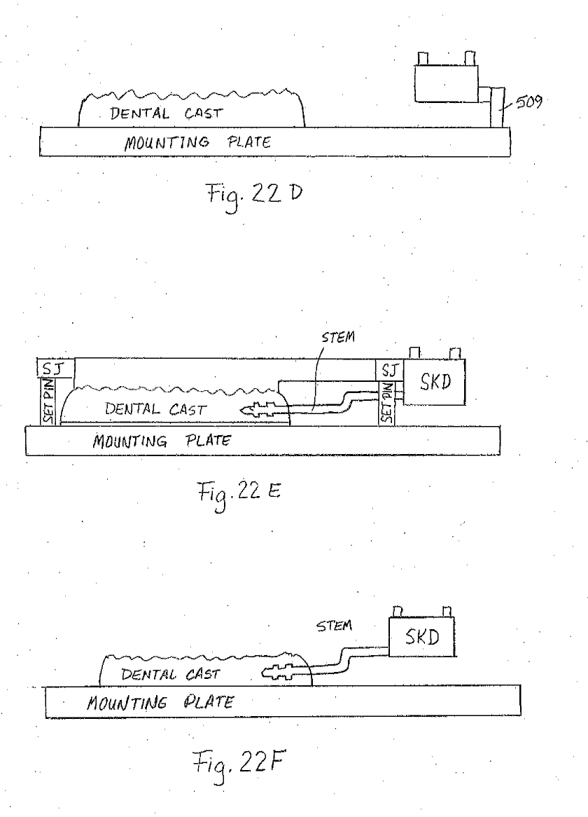

A further embodiment relates to the use of the radiographic template with a dental model to allow the attachment of a dental model to a base plate with the transfer of the identical position of the SKD to the baseplate so that a desk top scanner can be utilized to scan the dental cast for merger with the CT data set. FIG. 22A shows a radiographic appliance 500 having an impression is connected to an SKD 501. A dental model 503 is connected to the appliance 500 in FIG. 22B to create a model of the impression. The appliance is then mounted on a mounting plate 505 via set pins 507 and the model is connected to the mounting plate by a filler and/or adhesive. The SKD 501 is mounted to the mounting plate by an SKD mount 509 as shown in FIG. 22 D so that the positional relationship between the SKD and the impression is maintained between the SKD and the model 503 of the impression. As an alternative to the mount 509, the SKD may be mounted directly onto the model 503 as shown in FIGS. 22E and 22F.

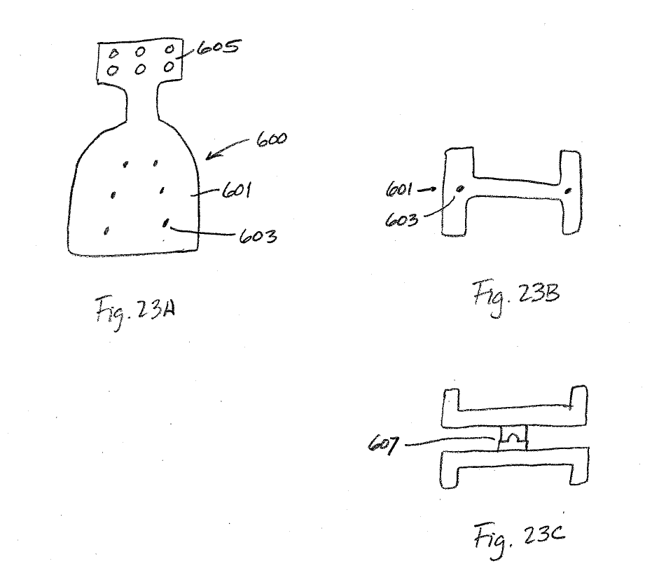

FIGS. 23A and 23B show a device 600 for incorporating the radiographic guides as a two part process of the upper and lower jaw arches to incorporate the CT data of the tooth roots in order to create the information of the opposing arches into the CT scan data base. An impression tray 601 that has polyvinylsiloxane to impression both of the upper and lower arches in a single bite is performed. Radiographic markers 603 are placed in the impression tray so that registration of the data set can be performed. An SKD 605 is attached to the handle of the tray so that there is a SKD facing each impression. Accordingly, there are two SKDs on opposing sides of the handle. The dual bite impression tray is removed from the patient and placed in a Styrofoam box and scanned in the CT scanner so that a second data set is obtained. The dual impression trays can be individual with a connecting member 607 for ease of removal from the mouth as shown in FIG. 23C. The dual impression radiographic guide data set is then registered with the CT scan data of the patient+radiographic guide. Optical scans of each negative impression is obtained which can then be registered with the CT data set via the SKD. Merger of the optical and CT data sets is performed. Articulation of the virtual dental models is performed and compared to clinical photos submitted by the dentist. Planning of the orthodontic movements is performed using the treatment planning software and a series of virtual dental models is created for each stage of orthodontic movement. This planning is innovative in that it also includes information about the tooth roots in the planning of the orthodontic tooth movements so as to improve the forces planned to move each tooth whether it is in rotation, tipping, or bodily movement based on known orthodontic biomechanical principles and at a certain velocity over time. Information concerning tooth roots relates to the avoidance of collisions between tooth roots during movements and to prevent the penetration and perforation of alveolar cortical bone so as to prevent gingival recession, mucogingival defects, and endodontic lesions. It would be anticipated that once this method is utilized commercially and a large number of cases performed particular information concerning the relationship of crown form to root form and their association with certain cases types that will aid in the planning and creation of a series of aligners for these cases. The following is an example of a production sequence in accordance with the present invention:

1. Impression of patient with special upper and lower jaw impression tray that has embedded radiographic markers and SKD.

2. Scan of the dual impression template

3. Optical scan of the negative impressions and SKD

4. Merger of data sets: Patient+Radiographic template, radiographic template and optical scan of the negative impressions of each arch and also to create an artifact corrected image when dental restorations or metallic orthodontic brackets are present.

5. If individual scans of each arch with a radiographic appliance is made different SKD can applied to each arch so as to allow merger of the different radiographic appliance with the optical scan of each negative impression.

6. Merger of registered data sets to create a virtual dental model with associated root anatomy.

7. Planning of orthodontic tooth movements which includes the mathematical calculation of the angulation of the planned tooth movement in tipping, rotation, intrusion, extrusion, or bodily movements so as to prevent root collision, penetration or perforation of the cortical bone from the initial to final position for the prevention of gingival recession, mucogingival defects, and endodontic lesions with calculation of the segmented tooth path for each individual tooth with calculation of the angulation of the tooth long axis and the segmented bony cortices.

8. Gingiva can be segmented out from the dental model for the orthodontic tooth movement planning and then reapplied when the series of models are created so that an anatomically correct aligner will be created.

9. Creation of a series of virtual dental models at each stage of the planned orthodontic tooth movement. This may include various hybrid elements such as elastic traction, TADs, and presence of orthodontic attachments for specific tooth movements and elastic traction, and attachments for retention. Such retention attachments can also include radiographic markers and SKD for initial/repeat CT scanning and optical scanning of the dentition for registration and merger of the Data. Other designs within the virtual dental model can be added that will create other biomechanical forces in the aligner.

10. Dental implant planning could be incorporated into such planning as well as any dental prosthetics for natural or implant teeth.

11. Once the planning is complete stereolithographic or rapid printed/rapid prototyped models of the teeth are created. Laser or other identification tags are placed on each model in sequence of aligner treatment.

12. On an assembly line the stereolithographic models are mounted on carriers and prepared to run down an assembly line. These carriers are located by RFD.

13. Webs of thermoplastic material are pressed over each stereolithographic model and aligners are created.

14. The aligner material hardens and can be trimmed by robotic CAD CAM milling to create the correct contours. Additional cut outs are created as specified in the dentist's prescription. Other grooves and contours can be milled in as well so as to create additional orthodontic biomechanical forces.

15. Aligners are then polished and sorted by sequence of treatment.

16. Aligners are then packaged according to prescription sequence and case.

17. Aligners are shipped to the dentist.

18. An alternative pathway is that based on the virtual model that aligners can be created virtually according to the sequence of planned orthodontic tooth movement and that they can be fabricated by rapid printing technology using suitable polymers that are suitable and FDA approved for long term presence in the oral cavity.

An additional embodiment is the creation of a virtual dental model as described above using the CT scan of the patient+dual aligner or single aligners, separate scan of the radiographic appliance(s), and merger via registration of the scan appliance. This application however relates to the placement of orthodontic brackets for a fixed orthodontic treatment. The planning software is used to plan the orthodontic treatment and what the final position of the teeth will be and the associated final orthodontic bracket position should be on each tooth so that brackets are located on the stereolithographic model and an aligner is created that will pick up the orthodontic brackets so that the aligner or appliance will be able to cement the orthodontic brackets on the teeth by an indirect technique. This is exemplified by the Cadent method disclosed in U.S. Pat. Nos. 6,099,314, 6,976,840, 7,147,464, and 7,738,989 that does not incorporate CT data or root anatomy into the treatment planning. According to the present invention, the root and alveolar bone cortical anatomy are segmented out and used to calculate the correct tooth angulations so as to prevent the penetration or perforation of bone so as to prevent the development of gingival recession, mucogingival defects, or endodontic lesions. The virtual model takes into account the full representation of the tooth from crown to root and the segmentation of the bone cortices for planning as described above with special consideration for the prevention of bony cortex penetration or perforation or endodontic devitalization and is therefore a novel improvement of this methodology.

As described above with reference to FIGS. 22A-22F, an embodiment of the present invention creates a dental model of the teeth with the transfer of the SKD in the exact position that it was in relation to the dentition. In this embodiment the radiographic appliance has the SKD attached via an attachment that allows the SKD handle to be detached and reattached to the base plate as it related exactly before to the dentition. In this way the dental model can be scanned if there are particular tooth anatomy that would preclude an accurate optical scan of the dental impression. This optical scan of the model can then be utilized to merge the dental model into the CT scan data which incorporates US Patent Application 20090113714. This mount that allows the transfer of the SKD to the dental cast may be a stem included within the plaster for retention rather than to the base mounting plate. The stem would have retentive elements to allow its retention within the plaster in a fixed position. A further embodiment would be a SKD that is held onto the impression tray handle by a male to female attachment so that the SKD is removable. The advantage of this is that the SKD can be reused after resterilization as it is a more expensive part and can reduce the costs of manufacturing the impression trays. The SKD can also have the optical scanning portion on opposite sides so that when the SKD is transferred to the holding stem to make it part of the dental cast that it is capable of being scanned from the same side as the dental cast occlusal surface and avoids the need for turning. There can be a dual handle or plurality of handles that have the SKD held by a male to female joint that allows it to be removable.

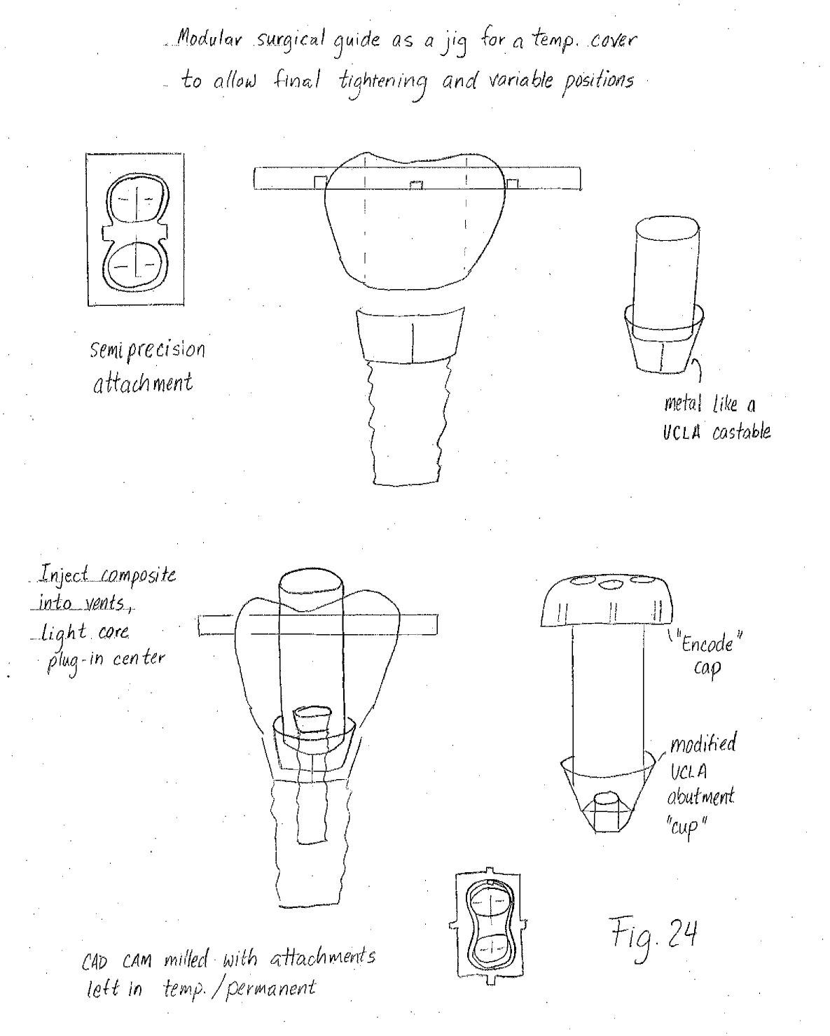

A further embodiment shown in FIG. 24 relates to the modular method of creating a CAD/CAM milled crown that will be inserted onto the dental implant in cases where the bone is less dense type II or III bone that may not allow the planned dental implant final position to be planned as precisely. As the implant is inserted into the bone using the method of US Application 20060291968 in a modular method with a modular fabricated drill guide it may be necessary to turn the implant several turns deeper into the bone so that the implant finally engages and locks into the final position into the bone. This precludes the placement of the CAD/CAM milled crown at the time of dental implant placement. An alternative is to have a CAD/CAM milled crown that will have an opening in the center that would accommodate a prefabricated post which could be straight or angled. The post is designed so that there is a widened base that extends into a cup form so that when the CAD/CAM milled crown is inserted onto the post, it will be attached by resin that is either cold or light cured. This cup then would catch any flowing resin and prevent it from getting into the bone or under the soft tissue or an undesirable aspect of the post base. The post can have a cap attached which could be as a snap on that would allow either an optical scan or traditional impression so that the data or model can be sent to a dental implant company such as Biomet 3i that would scan the model and create a custom final post and crown which would be inserted after a period of healing. It should be noted that the cup portion of the post that catches the flowing resin could be trimmed to create a correctly contoured temporary crown. The modular drill template can also act as a jig as the CAD/CAM milled temporary crown can have interlock attachments that would fit into the modular drill template framework and would allow the modular drill template gateway to act as a jig for the crown that would allow the crown to be placed in the planned and correct position to the post. Once placed the encode cap could also be placed and another optical scan or traditional impression obtained that relates the final dental implant position to the planned final CAD/CAM milled abutment or the crown as well. Such a temporary CAD CAM milled crown can be utilized as an orthodontic anchorage device that can be planned into the case based on the integrated CT and optical scan virtual model.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

What is claimed is:

1. A method for creating a dental model to avoid periodontal defects during planned dental work, comprising:
obtaining CT scan data and optical scan data of a patient's dentition;
integrating the CT scan data and the optical scan data by at least one of surface to surface registration, registration of radiographic markers, and registration of optical markers of known dimensions, to produce a dental model that includes the dentition and underlying bone and root structures;
determining segmented anatomic sites of the tooth roots and underlying bone;
generating a plan for the dental work based on the segmented anatomic sites, whereby the plan avoids periodontal defects based on the knowledge of the anatomic sites of the roots and underlying cortical bones in the dental model.

2. The method of claim 1, wherein the plan for the dental work is a plan for orthodontic tooth movements.

3. The method of claim 2, wherein thicknesses of the underlying cortical bones are taken into account, and the plan allows no more than a 50% penetration through the thicknesses of the cortical bones by the tooth roots during the orthodontic tooth movements.

4. The method of claim 2, relative positions of the roots and the cortical bones are displayed during the step of determining a plan.

5. The method of claim 1, further comprising the step of generating equipment to implement at least a portion of the dental work.

6. The method of claim 5, wherein the equipment is orthodontic equipment to implement planned tooth movements.

7. The method of claim 6, wherein the orthodontic equipment is orthodontic aligners.

8. The method of claim 5, wherein the equipment is a surgical guide.

9. The method of claim 8, wherein the step of obtaining CT scan data includes CT scanning a radiographic template including a module for the surgical guide, such that the dental model includes the position of the module relative to the tooth roots and the cortical bone.

10. The method of claim 9, further comprising the step of creating a drill guide sleeve in the module based on the position of the module relative to the tooth roots and the cortical bone after the step of generating a plan.

11. The method of claim 5, wherein the dental work includes orthognathic surgery, and the equipment generated is surgical splints.

12. The method of claim 5, wherein the equipment generated includes a dental implant.

13. The method of claim 5, wherein the equipment generated includes a dental prosthesis.

14. The method of claim 5, wherein the equipment generated includes a Temporary Anchorage Device (TAD).

15. The method of claim 1, wherein the step of obtaining comprises obtaining a CT scan of a radiographic template and a patient when the radiographic template is in a patient's mouth, the radiographic template having a negative impression of the patient's dentition and a surface of known dimensions, obtaining a CT scan of the radiographic template alone, and obtaining an optical scan of the radiographic template with the surface of known dimensions.

16. The method of claim 15, wherein the step of integrating comprises integrating the CT scan of the radiographic template and the patient with the CT scan of the radiographic template alone by registering radiographic markers, and integrating the optical scan and the CT scans by registering the surface of known dimensions.

17. The method of claim 16, wherein the radiographic template further includes a module to be used during dental surgery.

18. The method of claim 17, wherein the module is formed into a surgical template based on the anatomic sites of the roots and underlying cortical bones after the step of generating a plan.

19. The method of claim 17, wherein the module is connectable to and releasable from the template by interlocks and the surface of known dimensions is connectable to and releasable from one of the template and the module by further interlocks.

20. The method of claim 1, wherein the dental work comprises one of oral and maxillo facial surgery.

21. The method of claim 1, further including forming the dental model by one of stereolithography, rapid printing, and rapid prototyping.

22. The method of claim 1, wherein the dental model is a virtual model.
